(19) United States
(12) Patent Application Publication
(10) Pub. No.: US 2026/0179771 A1
(43) Pub. Date: Jun. 25, 2026

(54) SERVER FOR AUTOMATICALLY DIAGNOSING MALE INFERTILITY, AND METHOD FOR DIAGNOSING MALE INFERTILITY BY USING SAME

(71) Applicants: GOQBA TECHNOLOGY CORP., Seongnam-si (KR); SUNGKWANG MEDICAL FOUNDATION, Seoul (KR)

(72) Inventors: Jae Ho LEE, Yongin-si (KR); Hoon JIN, Suwon-si (KR); Jin Young PARK, Seoul (KR); Xia Jiun LAU, Seoul (KR); Sang Taek KONG, Pyeongtaek-si (KR); Moon Young SONG, Seoul (KR); In Hoo CHO, Bucheon-si (KR)

(73) Assignees: GOQBA TECHNOLOGY CORP., Seongnam-si (KR); SUNGKWANG MEDICAL FOUNDATION, Seoul (KR)

(21) Appl. No.: 19/127,953

(22) PCT Filed: Nov. 7, 2022
(86) PCT No.: PCT/KR2022/017380

§ 371 Date: Aug. 29, 2025

(51) Int. Cl. G16H 50/20; A61B 5/00; G16H 30/20

(52) U.S. Cl. CPC G16H 50/20; A61B 5/4375; G16H 30/20

(57) ABSTRACT

The present invention relates to a server for automatically diagnosing male infertility, and to a method for diagnosing male infertility by using the same and, more particularly, to a server that can diagnose male infertility through analysis of sperm motility trajectories and to a method for diagnosing male infertility by using the same. The present invention automates infertility diagnosis through analysis of sperm motility trajectories by using an artificial intelligence model, and can consistently diagnose even male infertility that cannot be diagnosed using existing male infertility analysis methods that inevitably reflect subjective standards. In addition, the present invention can provide automated analysis, and thus shows a significant improvement in analysis time and cost, and can be widely used in the male infertility diagnosis industry.

### TECHNICAL FIELD

The present disclosure relates to a male infertility automatic diagnosis server and a method for diagnosing male infertility by using the same.

### BACKGROUND ART

As the average age of marriage has recently increased, the proportion of infertile couples has increased significantly. In order to treat infertility, it is important to find the cause of infertility, so that an appropriate treatment method such as artificial insemination or in-vitro fertilization may be selected accordingly.

Among these cases of infertility, in the case of male infertility, in order to find the unknown cause through diagnosis, it is necessary to make a comprehensive judgment through collaboration between experts in basic research, clinical research, and artificial intelligence, to support the successful delivery of a healthy baby. The issue is that there are still limitations in diagnosing idiopathic male infertility patients who exhibit adequate sperm motility levels yet continue to experience infertility symptoms (Journal of Reproductive Immunology Volume 57, Issues 1-2, October-November 2002, Pages 35-45). In particular, assessing sperm motility through video observation is inherently limited by human vision, as it is extremely difficult to accurately analyze the motility patterns of 3,000 or more sperm cells simultaneously. Therefore, there is a need to develop technologies to overcome these limitations.

### DISCLOSURE

### Technical Problem

An aspect provides a male infertility automatic diagnosis system, including: a camera unit that captures sperm motility videos; a male infertility automatic diagnosis server; and a cloud platform for transmitting and receiving data with the server,
wherein the male infertility automatic diagnosis server includes: a data collection unit that obtains sperm motility videos from the camera unit and the cloud platform; a data preprocessing unit that performs preprocessing on video data obtained by the data collection unit; a machine learning unit that inputs the sperm motility video obtained from the cloud platform into an input node of an artificial intelligence model, and inputs male infertility diagnosis results obtained from the cloud platform into an output node of the artificial intelligence model to perform training; an infertility determination unit that receives the preprocessed video data and determines whether male infertility is present; and a data providing unit that provides the male infertility diagnosis results to the cloud platform.

Another aspect provides a method for diagnosing male infertility by using the male infertility automatic diagnosis system, the method including:
obtaining, by a male infertility automatic diagnosis server, sperm motility videos and data on male infertility from a cloud platform; inputting, by the male infertility automatic diagnosis server, sperm motility videos into an input node of an artificial intelligence model and inputting a male infertility status into an output node of an artificial intelligence model to perform training; capturing, by the camera unit, a video of sperm motility; obtaining, by the male infertility automatic diagnosis server, the video of sperm motility captured by the camera unit; preprocessing, by the male infertility automatic diagnosis server, the obtained sperm motility video; inputting, by the male infertility automatic diagnosis server, the preprocessed sperm motility video into the trained artificial intelligence model to determine whether male infertility is present; and providing, by the male infertility automatic diagnosis server, the male infertility diagnosis data to the cloud platform.

However, the problems to be solved by the present invention are not limited to the problems mentioned above, and other problems not mentioned may be clearly understood by a person having ordinary skill in the relevant technical field from the description below.

### Technical Solution

An aspect may be a male infertility automatic diagnosis system, including: a camera unit that captures sperm motility videos; a male infertility automatic diagnosis server; and a cloud platform for transmitting and receiving data with the server,
wherein the male infertility automatic diagnosis server includes: a data collection unit that obtains sperm motility videos from the camera unit and the cloud platform; a data preprocessing unit that performs preprocessing on the video data obtained by the data collection unit; a machine learning unit that inputs the sperm motility video obtained from the cloud platform into an input node of an artificial intelligence model, and input male infertility diagnosis results obtained from the cloud platform into an output node of the artificial intelligence model to perform training; an infertility determination unit that receives the preprocessed video data and determines whether male infertility is present; and a data providing unit that provides the male infertility diagnosis results to the cloud platform.

In an embodiment, the machine learning unit may be perform training using a machine learning technique or a deep learning technique.

Another aspect may be a method for diagnosing male infertility using the male infertility automatic diagnosis system, the method including:
obtaining, by the male infertility automatic diagnosis server, sperm motility videos and data on male infertility from the cloud platform; inputting, by the male infertility automatic diagnosis server, sperm motility videos into an input node of an artificial intelligence model and inputting the male infertility status into an output node of an artificial intelligence model to perform training; capturing, by the camera unit, a video of the sperm motility; obtaining, by the male infertility automatic diagnosis server, the video of sperm motility captured by the camera unit; preprocessing, by the male infertility automatic diagnosis server, the obtained sperm motility video; inputting, by the male infertility automatic diagnosis server, the preprocessed sperm motility video into the trained artificial intelligence model to determine whether male infertility is present; and providing, by the male infertility automatic diagnosis server, the male infertility diagnosis data to the cloud platform.

In an embodiment, the processing the sperm motility video may include extracting the sperm motility trajectory; and classifying sperm motility patterns.

In an embodiment, the determining whether male infertility is present may include deriving a class, number, speed and motility distance of the sperm.

In an embodiment, the training may be training using a machine learning technique or a deep learning technique.

### Advantageous Effects

The present invention automates the diagnosis of male infertility through analysis of sperm motility trajectories using an artificial intelligence model. As such, it enables consistent diagnosis of male infertility cases that could not be diagnosed by conventional male infertility analysis methods, which inherently relied on subjective criteria. Furthermore, because it provides automated analysis, it significantly improves both the time and cost efficiency of the diagnostic process, and thus may be widely utilized in the male infertility diagnostic industry.

### DESCRIPTION OF DRAWINGS

FIG. 1 is a block diagram of a system of the present invention.

FIG. 2A is a flowchart showing a comprehensive male infertility diagnosis method.

FIG. 2B is a flowchart showing a method of performing video data preprocessing in the diagnosis method of FIG. 2A.

FIG. 3A is a video image of a data preprocessing operation in the male infertility diagnosis method of the present invention, showing the sperm motility pattern and the sperm motility trajectory classified by color through CASA video capture.

FIG. 3B is a video image of a data preprocessing operation in the male infertility diagnosis method of the present invention, showing the sperm motility trajectory divided by sperm motility pattern.

FIG. 3C is a video image of a data preprocessing operation in the male infertility diagnosis method of the present invention, showing extracted individual sperm motility trajectories.

FIG. 4 is a diagram showing a method for object recognition and motility pattern prediction by using an artificial intelligence model.

### BEST MODE

In describing the present disclosure, description of technical contents that are well known in the technical field to which the present disclosure belongs and are not directly related to the present disclosure will be omitted. This is to convey the gist of the present disclosure more clearly without obscuring it by omitting unnecessary explanations. And the terms described below are terms defined in consideration of the functions in the present disclosure, and may vary depending on the intention or custom of the user or operator. Therefore, the definition should be based on the contents throughout this specification.

For the same reason, some components in the attached drawings are exaggerated, omitted, or schematically depicted. Additionally, the size of each component does not entirely reflect its actual size. In each drawing, identical or corresponding components are given the same reference numbers.

The advantages and features of the present disclosure and the methods for achieving them will become apparent by reference to the embodiments described below in detail together with the accompanying drawings. However, the present disclosure is not limited to the embodiments disclosed below and may be implemented in various different forms. The disclosed embodiments are provided so that this disclosure will be thorough and complete, and will fully convey the scope of the disclosure to those skilled in the art to which this disclosure pertains. An embodiment of the present disclosure may be defined by the claims. Identical reference numerals throughout the specification represent identical components. In addition, when describing an embodiment of the present disclosure, if it is determined that a detailed description of a related function or configuration may unnecessarily obscure the gist of the present disclosure, the detailed description will be omitted. And the terms described below are terms defined in consideration of the functions in the present disclosure, and may vary depending on the intention or custom of the user or operator. Therefore, the definition should be based on the contents throughout this specification.

In an embodiment, each block of the flowchart drawings and combinations of the flowchart drawings may be performed by computer program instructions. The computer program instructions may be mounted on a processor of a general-purpose computer, a special-purpose computer, or other programmable data processing apparatus, and the instructions executed by the processor of the computer or other programmable data processing apparatus may generate means for performing the functions described in the flowchart block(s). The processor may write data to memory or read data stored in memory, and in particular, may process data according to predefined operation rules or an artificial intelligence model by executing a program stored in the memory. Therefore, the processor may perform the operations described in the embodiments that follow, and the operations described as being performed by the server in the embodiments that follow may be regarded as being performed by the processor unless otherwise specified. The computer program instructions may also be stored in a computer-usable or computer-readable memory that may direct a computer or other programmable data processing apparatus to implement functions in a specific manner, and the instructions stored in the computer-usable or computer-readable memory may produce an article of manufacture including instruction means for performing the functions described in the flowchart block(s). The computer program instructions may also be mounted on a computer or other programmable data processing apparatus.

The memory is a configuration for storing various programs or data, and may be composed of a storage medium such as ROM, RAM, hard disk, CD-ROM, and DVD, or a combination of such storage media. The memory may not exist separately and may be configured to be included in the processor. The memory may be composed of volatile memory, non-volatile memory, or a combination of volatile memory and non-volatile memory. A program for performing the operations according to the embodiments described later may be stored in the memory. The memory may also provide the stored data to the processor in response to a request from the processor.

Also, each block of the flowchart diagrams may represent a module, segment, or portion of code that includes one or more executable instructions for performing the specified logical function(s). In an embodiment, the functions mentioned in the blocks may occur out of order. For example, two blocks shown in succession may be executed substantially simultaneously, or in reverse order depending on the functions.

The term “unit” as used in an embodiment of the present disclosure may refer to a software component or a hardware component such as an FPGA (Field Programmable Gate Array) or an ASIC (Application Specific Integrated Circuit), and the “unit” may perform a specific function. Meanwhile, the term “˜unit” is not limited to software or hardware. The “˜unit” may be configured to exist in an addressable storage medium and may be configured to reproduce one or more processors. In an embodiment, the “˜unit” may include components such as software components, object-oriented software components, class components, and task components, as well as processes, functions, properties, procedures, subroutines, segments of program code, drivers, firmware, microcode, circuits, data, databases, data structures, tables, arrays, and variables. The functions provided through specific components or specific “˜units” may be combined to reduce the number of components or may be separated into additional components. Also, in an embodiment, the “˜unit” may include one or more processors.

Hereinafter, embodiments of the present disclosure will be described in detail with reference to the attached drawings.

The terms “includes” or “comprises” used in this specification should not be construed to necessarily include all of the components or operations described in the specification, and some of the components or operations may not be included, or may include additional components or operations.

Each description should not be construed as limiting the scope of the rights, and what can be easily inferred by a person skilled in the relevant technical field should be interpreted as falling within the scope of the rights.

Hereinafter, embodiments of the present disclosure will be described in detail with reference to the attached drawings.

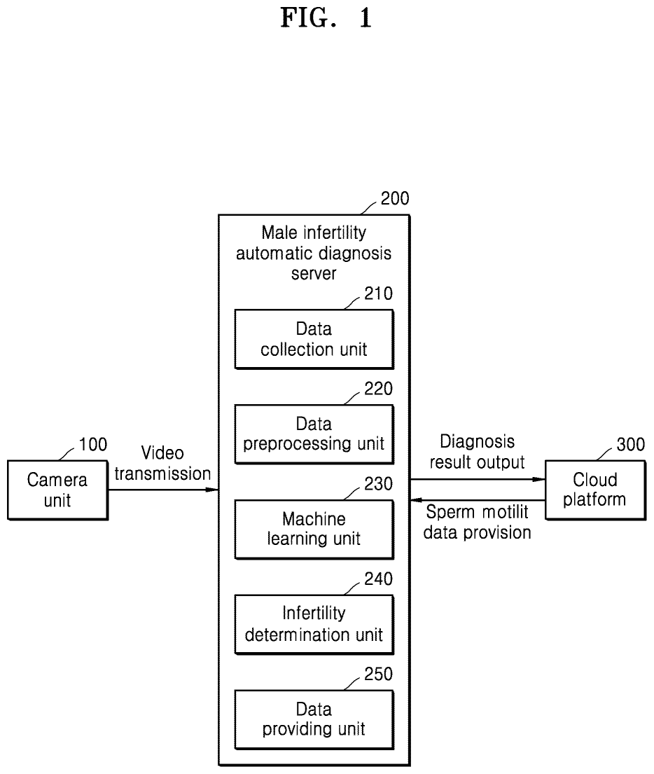

FIG. 1 illustrates a block diagram of a male infertility automatic diagnosis system according to an embodiment of the present disclosure.

Referring to FIG. 1, the male infertility automatic diagnosis system according to an embodiment of the present invention may include a camera unit 100, a male infertility automatic diagnosis server 200, and a cloud platform 300, and the male infertility automatic diagnosis server may include a data collection unit 210, a data preprocessing unit 220, a machine learning unit 230, an infertility determination unit 240, and a data providing unit 250.

In the present specification, the term “male infertility” refers to a condition in which pregnancy does not occur despite having regular sexual intercourse without contraception over a certain period of time, and male infertility may be classified into categories such as sperm abnormalities, obstruction of the sperm transport pathway, semen abnormalities such as accessory gland dysfunction, erectile dysfunction, and ejaculation disorders. In the case of sperm abnormalities, it may be due to an abnormality in the number, shape, or motility of the sperm, impaired sperm production in the testes, cryptorchidism, varicocele, hydrocele of the scrotum, or exposure to toxic substances. In the case of obstruction of the sperm transport pathway, it may be due to abnormalities in the testes, epididymis, vas deferens, or ejaculatory ducts, or due to conditions such as epididymitis, congenital absence of the vas deferens, prostatitis, or obstruction of the vas deferens. Semen abnormalities may be due to dysfunction of the seminal vesicles and prostate, which are responsible for the storage and quality of semen, but are not limited thereto.

The camera unit 100 according to an embodiment of the present invention may capture video of the motility state of the sperm. In addition, although the camera unit is not limited as long as it captures video of the motility state of the sperm, it may be a CASA (Computer Assisted Semen Analysis) used for capturing sperm motility.

The male infertility automatic diagnosis server 200 according to an embodiment of the present invention may receive sperm motility video data from the cloud platform 300 to perform training of an artificial intelligence model and may acquire sperm motility video data from the camera unit 100 to determine whether male infertility is present. The male infertility automatic diagnosis server 200 may be connected to the camera unit 100 and the cloud platform 300 via wireless or wired communication, and the communication method is not particularly limited and may include, for example, 3G, LTE, 5G, Wi-Fi, or Bluetooth.

The male infertility automatic diagnosis server 200 includes a data collection unit 210 that obtains sperm motility videos from the camera unit and the cloud platform; a data preprocessing unit 220 that performs preprocessing on the video data obtained by the data collection unit; a machine learning unit 230 that inputs the sperm motility video acquired from the cloud platform into the input node of an artificial intelligence model and inputs the male infertility diagnosis result obtained from the cloud platform into the output node of the artificial intelligence model to perform training; an infertility determination unit 240 that receives the preprocessed video data and determines whether male infertility is present; and a data providing unit 250 that provides the male infertility diagnosis result to the cloud platform.

The data collection unit 210 may obtain sperm motility videos from the camera unit 100, or may obtain sperm motility data and corresponding male infertility diagnosis data from the cloud platform 300.

The data preprocessing unit 220 may receive sperm motility video data from the data collection unit 210 and perform preprocessing.

The machine learning unit 230 may receive the sperm motility video acquired from the cloud platform from the data collection unit, input it into the input node of the artificial intelligence model, and input the male infertility diagnosis result acquired from the cloud platform into the output node of the artificial intelligence model to perform training. The training may be performed using one or more selected from the group consisting of machine learning (for example, Support Vector Machine SVM, Artificial Neural Network ANN, Recurrent Neural Network RNN) and deep learning (for example, Deep Neural Network DNN).

The infertility determination unit 240 may receive the preprocessed video data from the data preprocessing unit 220 and determine whether male infertility is present.

The cloud platform 300 provides computing services such as servers, storage, databases, networking, software, analytics, and intelligence via the Internet (“cloud”).

The cloud platform 300 may store data for male infertility testing (including sperm motility videos, and other data related to dementia, etc.) from around the world via the Internet, and may provide such data to the male infertility automatic diagnosis server 200 upon request, and may receive and store male infertility diagnosis results from the male infertility automatic diagnosis server.

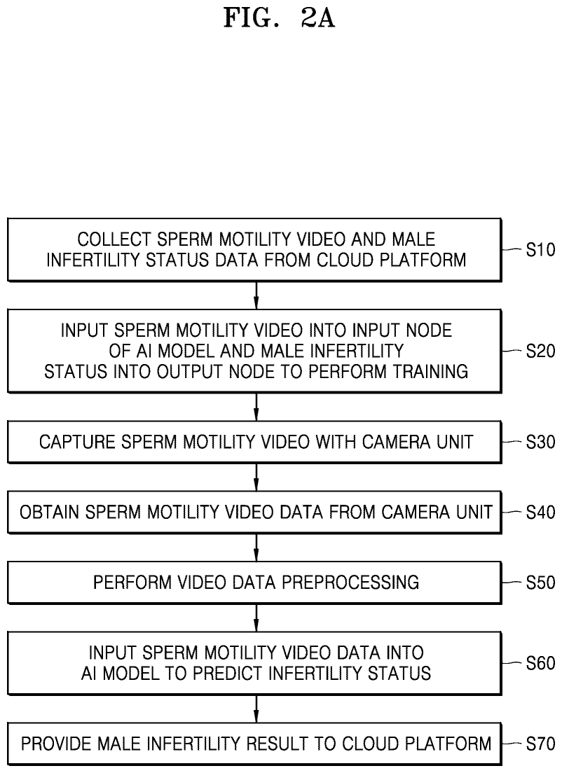

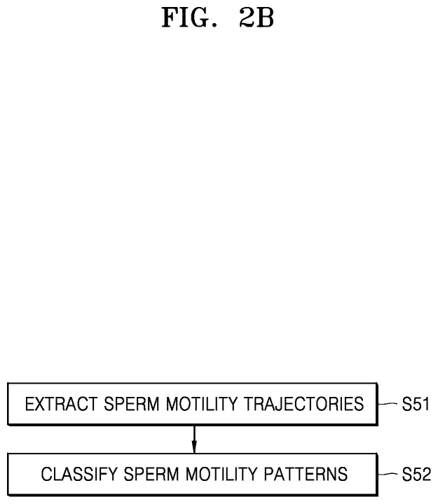

FIG. 2 is a flowchart illustrating a male infertility diagnosis method of the system according to an embodiment of the present disclosure, wherein S denotes Step.

Referring to FIG. 2, the male infertility diagnosis method according to an embodiment of the present invention includes: an operation S10 in which the male infertility automatic diagnosis server acquires sperm motility videos and data regarding male infertility from the cloud platform; an operation S20 in which the male infertility automatic diagnosis server inputs the sperm motility video into the input node of an artificial intelligence model and inputs the male infertility status into the output node of the artificial intelligence model to perform training; an operation S30 in which the camera unit captures a video of sperm motility; an operation S40 in which the male infertility automatic diagnosis server obtains the sperm motility video captured by the camera unit; an operation S50 in which the male infertility automatic diagnosis server performs preprocessing on the obtained sperm motility video; an operation S60 in which the male infertility automatic diagnosis server inputs the preprocessed sperm motility video into the trained artificial intelligence model to determine whether male infertility is present; and an operation S70 in which the male infertility automatic diagnosis server provides the male infertility diagnosis data to the cloud platform.

The male infertility diagnosis method according to an embodiment of the present invention may apply the contents described above with respect to the male infertility automatic diagnosis system. Therefore, for the male infertility diagnosis method, identical content to the above-described male infertility automatic diagnosis system is omitted.

The operation S30 of capturing sperm motility video by the camera unit is not limited as long as it captures a video of sperm motility, but as described above, it may be a video captured by CASA, and the video captured by CASA may classify the sperm trajectories into A, B, C, or D according to sperm motility patterns.

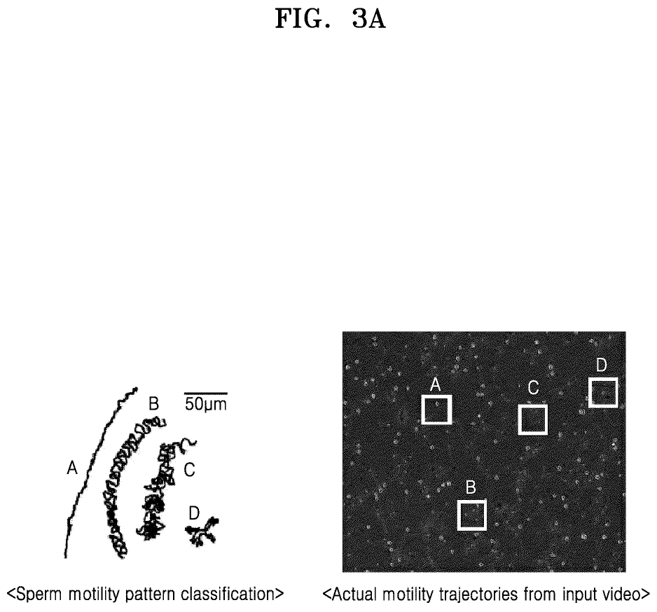

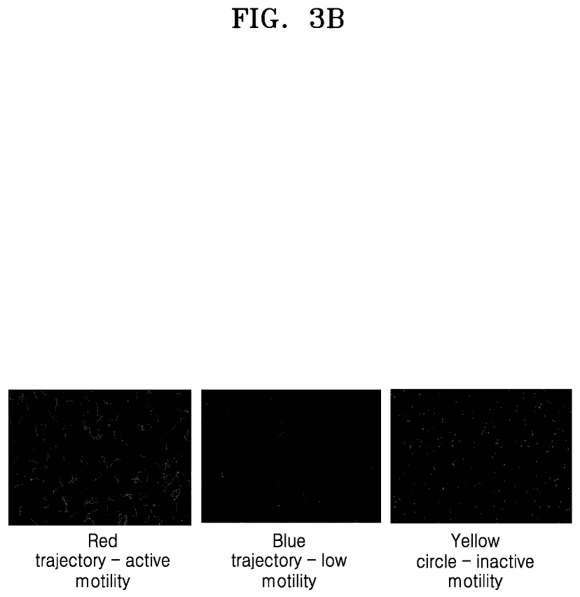

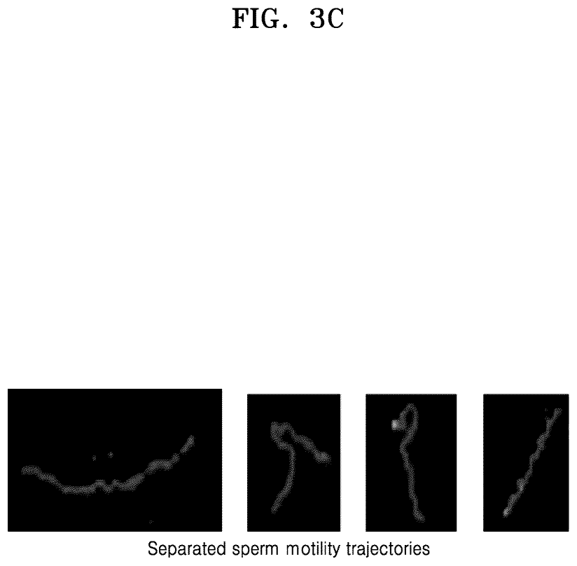

As shown in FIG. 3A, the sperm motility patterns may be classified into A, B, C, or D. Specifically, classification A refers to a type with linear motility, meaning that the sperm movement exhibits a straight-line form. Classification B means that the motility of the sperm shows weak linearity due to rotation compared to straight-line motility. Classification C means that the sperm exhibits strong rotational movement and does not show linearity. Classification D means that the sperm performs non-directional rotational movement, rotating around its original position.

In the case of capturing sperm motility videos using CASA, as shown in FIG. 3B, according to the classification, classification A is indicated by a red trajectory, classification B is indicated by a blue trajectory, and classification C is indicated by a yellow circle.

The operation S50 of preprocessing the sperm motility video may include an operation S51 of extracting sperm motility trajectories, and an operation S52 of classifying sperm motility patterns. The operation S51 of extracting motility trajectories may be an operation of extracting the trajectories representing the movement of each individual sperm, as shown in FIG. 3C.

The operation S52 of classifying sperm motility patterns may be performed according to Mathematical Equation 1 below. The trajectory information of sperm motility may be derived such that the closer the directionality is to the positive direction, the closer the value is to 1; the more correlated to the negative direction, the closer the value is to −1; and the closer the value is to 0, the less correlation there is with the movement direction.

$$PCC = \frac{{\sum }_{i}^{n} ( {X}_{i} - \overline{X} ) ( {Y}_{i} - \overline{Y} )}{\sqrt{{\sum }_{i}^{n} {( {X}_{i} - \overline{X} )}^{2}} \sqrt{{\sum }_{i}^{n} {( {Y}_{i} - \overline{Y} )}^{2}}} \qquad [ Mathematical Formula 1 ]$$

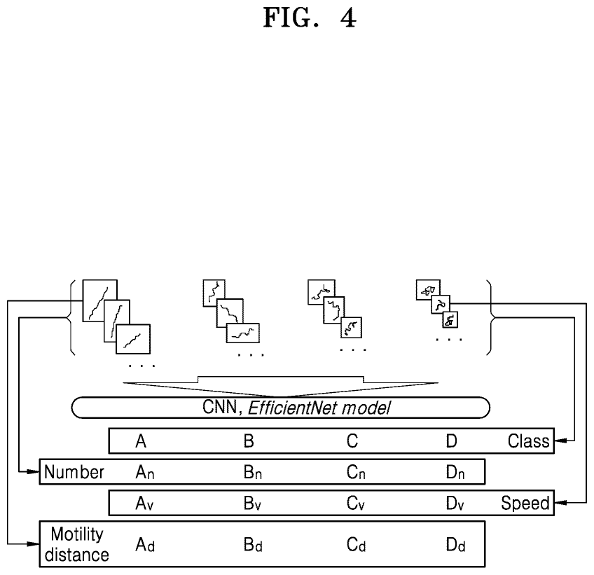

The operation S60 in which the male infertility automatic diagnosis server inputs the preprocessed sperm motility video into the trained artificial intelligence model to determine whether male infertility is present includes the operation of deriving the sperm class (classification), the number by class, the sperm speed, and the sperm motility distance, as shown in FIG. 4.

When using the male infertility automatic diagnosis method, chromosomal abnormalities of the sperm may also be determined.

1. A male infertility automatic diagnosis system, comprising:
a camera unit that captures sperm motility videos;
a male infertility automatic diagnosis server; and
a cloud platform for transmitting and receiving data with the server,
wherein the male infertility automatic diagnosis server comprises:
a data collection unit that obtains sperm motility videos from the camera unit and the cloud platform;
a data preprocessing unit that performs preprocessing on video data obtained by the data collection unit;
a machine learning unit that inputs the sperm motility videos obtained from the cloud platform into an input node of an artificial intelligence model, and inputs male infertility diagnosis results obtained from the cloud platform into an output node of the artificial intelligence model to perform training;
an infertility determination unit that receives the preprocessed video data and determines whether male infertility is present; and
a data providing unit that provides the male infertility diagnosis results to the cloud platform.

2. The system of claim 1,
wherein the machine learning unit performs training using a machine learning technique or a deep learning technique.

3. A method for diagnosing male infertility by using the male infertility automatic diagnosis system of claim 1, the method comprising:
obtaining, by a male infertility automatic diagnosis server, sperm motility videos and data on male infertility from a cloud platform;
inputting, by the male infertility automatic diagnosis server, sperm motility videos into an input node of an artificial intelligence model and inputting a male infertility status into an output node of an artificial intelligence model to perform training;
capturing, by the camera unit, a video of sperm motility;
obtaining, by the male infertility automatic diagnosis server, the video of sperm motility captured by the camera unit;
preprocessing, by the male infertility automatic diagnosis server, the obtained sperm motility video;
inputting, by the male infertility automatic diagnosis server, the preprocessed sperm motility video into the trained artificial intelligence model, to determine whether male infertility is present; and
providing, by the male infertility automatic diagnosis server, the male infertility diagnosis data to the cloud platform.

4. The method of claim 3,
wherein the preprocessing of the sperm motility video comprises:
extracting sperm motility trajectory; and
classifying sperm motility patterns.

5. The method of claim 3,
wherein the determining of whether male infertility is present comprises deriving a class, number, speed and motility distance of sperm.

6. The method of claim 3,
wherein the training is performed using a machine learning technique or a deep learning technique.
